(19) United States
(12) Patent Application Publication
(10) Pub. No.: US 2005/0187196 A1
(43) Pub. Date: Aug. 25, 2005

(54) Treatment of attention disorders

(75) Inventors: Annette Madrid, El Granada, CA (US); Helen Jenkins, Foster City, CA (US); Rodney Pearlman, El Granada, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Saegis Pharmaceuticals, Inc., Half Moon Bay, CA (US)

(21) Appl. No.: 11/064,887

(22) Filed: Feb. 23, 2005

Related U.S. Application Data
(60) Provisional application No. 60/547,371, filed on Feb. 23, 2004.

(51) Int. Cl. A61K 31/66; A61K 31/445; A61K 31/137

(52) U.S. Cl. 514/114; 514/317; 514/649

(57) ABSTRACT

The invention provides methods and medicaments for improving attentiveness in humans, including subjects diagnosed with attention disorders. In one aspect, a GABA_{B} receptor antagonist, such as 3-aminopropyl-(n-butyl)-phosphinic acid (ABPA), is used to improve attention.

### CROSS-REFERENCE TO RELATED APPLICATIONS

This application claims benefit of provisional application No. 60/547,371, filed Feb. 23, 2004, the entire contents of which are incorporated herein by reference.

### FIELD OF THE INVENTION

The invention provides methods and medicaments for improving attentiveness in humans, including subjects diagnosed with attention disorders. The invention has application in the fields of neurobiology, neuropsychology, and medicine.

### BACKGROUND

Attention deficit is a primary component of attention-deficit/hyperactivity disorders (ADHD) that affect a significant portion of the population. For example, it has been estimated about three to seven percent of children suffer from ADHD. More recently, studies have demonstrated that ADHD persists into adulthood for as many as 60% of individuals affected with the disorder in childhood. ADHD is now seen as a condition that affects a large proportion of the pediatric and adult population, many of whom can benefit from pharmacological intervention. The primary pharmacological treatments for this disorder are psychostimulant drugs, such as methylphenidate (Ritalin®) and amphetamine formulations, which in general have been effective in producing behavioral improvements. A significant proportion of ADHD patients, however, either do not respond to stimulants, cannot tolerate their side effects such as nausea and insomnia, or cannot use them because of their abuse potential. Due to these many factors, non-stimulant agents with anti-ADHD efficacy are needed.

### BRIEF SUMMARY OF THE INVENTION

The invention relates generally to methods and compositions for enhancing attention in a mammal, particularly a human subject. In one aspect, the invention provides a method for treatment of an attention disorder by administering an effective amount of a GABAB receptor antagonist to a subject in need of such treatment. In a preferred embodiment, the antagonist is 3-aminopropyl-(n-butyl)-phosphinic acid (ABPA). Attention disorders susceptible to treatment include Attention Deficit Disorder (ADD) and Attention-Deficit/Hyperactivity Disorder (ADHD). In a related aspect the invention provides the use of a GABA_{B} receptor antagonist (such as 3-aminopropyl-(n-butyl)-phosphinic acid) in the preparation of a medicament for treatment of Attention Deficit Disorder (ADD) or for treatment of Attention-Deficit/Hyperactivity Disorder (ADHD).

In various embodiments of the invention the subject to whom treatment is administered is (a) an adult, (b) a child, (c) a subject not diagnosed with Mild Cognitive Impairment (MCI), or (d) a subject not diagnosed with Alzheimer's Disease (AD).

In one embodiment of the invention the GABAB receptor antagonist is administered in combination with a second treatment for attention deficit, such as, but not limited to, dextroamphetamine, methylphenidate, pemoline or atomoxetine hydrochloride.

In an aspect, the invention provides a method for treatment comprising administering a therapeutically effective amount of a GABA_{B} receptor antagonist to a subject in need of treatment for ADHD or ADD. In a related aspect the invention provides a method for treatment comprising (a) diagnosing a disorder of attention in a subject, (b) administering a GABA_{B} receptor antagonist to the subject and (c) determining whether one or more manifestations of the disorder are reduced in the subject. In a related aspect, the invention provides a method for treatment comprising (a) administering a GABA_{B} receptor antagonist to the subject diagnosed with a disorder of attention and (b) determining whether one or more manifestations of disorder are reduced in the subject. In yet another related aspect, the invention provides a method for treatment comprising (a) diagnosing an attention disorder in a subject, (b) administering a GABA_{B} receptor antagonist to the subject for a period of time, and (c) detecting an improvement in a quantitative measure of attention following step (b). In yet another related aspect, the invention provides a method for treatment comprising (a) administering a GABA_{B} receptor antagonist for a period of time to a subject diagnosed with an attention disorder and (b) detecting an improvement in a quantitative measure of attention following step (a).

In another aspect, the invention provides a method for identification of an agent useful for treatment of an attention disorder by (1) identifying an agent as a GABAB receptor antagonist and (2) determining whether the antagonist has an attention enhancing effect in a mammal.

### BRIEF DESCRIPTION OF THE FIGURES

FIG. 1 shows the improvement in mean reaction time in the Five Choice Reaction Time test in patients diagnosed with mild cognitive impairment (MCI) administered ABPA (600 mg tid) for eight weeks. The plot shows mean reaction time (in milliseconds) for ABPA- and placebo-treated patients at four testing time points: Baseline and after 2, 5 and 8 weeks of treatment. Significant differences (*, p<0.05) in performance between the two treatment groups were seen at 8 weeks, while trends (#, p<0.10) were present during testing at week 2 and week 5.

FIG. 2 shows the improvement in mean hit latency in the Rapid Visual Information Processing test in patients diagnosed with MCI administered ABPA (600 mg tid) for eight weeks. The plot shows mean hit latency (in milliseconds) for ABPA- and placebo-treated patients at the four time points tested. Significant differences (*, p<0.05) in performance between the two groups were seen at 5 weeks and 8 weeks of treatment.

FIG. 3 shows the hyperactivity of coloboma mutant mice and the attenuating effect of ABPA. The plots in FIGS. 3A and 3B show the total distance traveled (in centimeters) by coloboma and wildtype mice in the final 30 minutes of a 60-minute session in the open field arena. In FIG. 3A, treatment with 0.1 mg/kg atomoxetine significantly reduced the spontaneous hyperactivity exhibited by the coloboma mice (*, p<0.05). In FIG. 3B, three different doses of ABPA (0.3, 3, and 30 mg/kg) were effective in reducing the heightened activity of coloboma mice. This reduction was statistically significant for the two highest doses (*, p<0.05) while the low dose produced a clear trend (#, p<0.10).

FIG. 4 shows the hyperactivity of coloboma mutant mice and the normalizing effect of ABPA. The plots in FIGS. 4A and 4B show the frequency of zone crossings by coloboma and wildtype mice in the final 30 minutes of a 60-min session in the open field arena. In FIG. 4A, treatment with 0.1 mg/kg atomoxetine produced a marked trend towards reduced locomotor activity in the coloboma mice (#, p<0.10). In FIG. 4B, three different doses of ABPA (0.3, 3, and 30 mg/kg) were also effective in reducing the frequency of zone crossings by coloboma mice. This reduction was statistically significant for the high and low doses of ABPA (*, p<0.05) while the middle dose produced a clear trend towards reduced locomotor activity (#, p<0.10).

### DETAILED DESCRIPTION

I. Introduction

It has been discovered that, surprisingly, improvement is observed in parameters of attention when a gamma-aminobutyric acid B (GABA_{B}) receptor antagonist is administered to human subjects. These results indicate that compounds with GABA_{B} receptor antagonist activity can be administered to treat patients for whom improved attention is desirable, such as patients with attention disorders. In particular, 3-aminopropyl-(n-butyl)-phosphinic acid (ABPA) is useful for treatment of attention disorders. Further, administration of ABPA reduced spontaneous hyperactivity in a mouse model of Attention-Deficit/Hyperactivity Disorder, indicating that this and other GABA_{B} receptor antagonists are also effective in treating the hyperactivity component of the disorder.

II. Attention Disorders

In one aspect, the invention provides methods and compositions useful for treatment of attention disorders and for enhancement of attention generally. Attention disorders are conditions characterized by impaired ability to concentrate on selected features of the environment to the relative exclusion of others. For example and not for limitation, such disorders include Attention Deficit Disorder (ADD) and Attention-Deficit/Hyperactivity Disorder (ADHD).

The disorders described herein are well known, and thus are only briefly described below. It is within the skill of medical professionals to diagnose such disorders with reference to the medical literature, and thereby identify individuals with a disorder. Diagnostic criteria are found in (1) DIAGNOSTIC AND STATISTICAL MANUAL OF MENTAL DISORDERS (4th Edition, American Psychiatric Association (hereinafter“DSM-IV”)) incorporated by reference herein; (2) The International Statistical Classification of Diseases and Related Health Problems, Tenth Revision (hereinafter “IDC-10”) incorporated by reference herein; (3) the medical literature.

Attention Deficit Disorder

Attention deficit disorder (ADD) is a disorder that affects children, adolescents and adults. Subjects suffering from the disorder typically have difficulty concentrating, listening, learning and completing tasks; and are restless, fidgety, impulsive and easily distracted. The diagnostic criteria for ADD are provided in the DSM-IV.

Attention Deficit Hyperactivity Disorder

Attention-deficit/hyperactivity disorder (ADHD) includes the symptoms of ADD as well as a high level of activity (e.g., restlessness and movement). ADHD is a common disorder of childhood, with a prevalence of 3-7% of the general population. Diagnostic criteria for ADHD are provided in the DSM-IV. Although ADHD and ADD are characterized by different diagnostic criteria (e.g., including the manifestation of hyperactivity in ADHD) the biological basis of the attention deficit is likely the same in the two conditions.

III. Gamma-Aminobutyric Acid Receptor Antagonists

A variety of GABA_{B} receptor antagonists may be used in the methods of the invention. 3-Aminopropyl-(n-butyl)-phosphinic acid (“ABPA”) is a particularly preferred antagonist and has been shown to have an effect on attentional processes in humans and animals (see Examples below). Other GABA_{B} receptor antagonists include, for example 3-{1(S)-[3-(cyclohexylmethyl) hydroxyphosphinyl)-2(S)-hydroxy-propylamino]ethyl}benzoic acid; 3-{1(R)-[3-(cyclohexylmethyl) hydroxyphosphinyl-2(S)-hydroxy-propylamino]ethyl}benzoic acid; other phosphinic acid analogues including, without limitation, CGP27492, CGP35024, CGP47656, CGP36216, CGP35348, CGP35913, phaclophen and others such as those described in Froestl et al., 2003, “Ligands for expression cloning and isolation of GABAb receptors” II Farmaco 58:173-83 and Enna, 1997, “GABA_{B} receptor agonists and antagonists: pharmacological properties and therapeutic possibilities” Exp Opin Invest Drugs 6:1319-1325; 2,5 disubstituted-1,4-morpholines (see, e.g., Bolser et aL, 1995, “The pharmacology of SCH-50911: a novel, orally-active GABA-B receptor antagonist” JPET 274:1393-1448); benzyl-substituted phosphinic acids including, without limitation, CGP54626A, CGP62349, CGP54748A, CGP57076A, CGP67588, CGP80936 and others such as those described in Froestl et al., supra and Enna, supra; and compounds described in Bittiger et al., 1993, Trends Pharmacol Sci 1993:14:391-393; Olpe et al., 1990, “CGP 35348: a centrally active blocker of GABA_{B} receptors” Eur J Pharmacol. 187:27-38; patent publication US20020091250A1 entitled “Metabotropic GABA [B] receptors, receptor-specific ligands and their uses;” patent publication WO 04000326A1 entitled “Combination therapy wherein a serotonin reuptake inhibitor is used”; and U.S. Pat. Nos. 5,300,679 and 5,064,819 entitled “Substituted propane-phosphinic acid compounds.” It will be appreciated that GABA_{B} receptor antagonists may also be antagonize other GABA receptors; for example, ABPA is reported to antagonize the GABA_{C} receptor (see U.S. Pat. No. 6,632,806 “Neurologically-active compounds”). In a related embodiment, agents with GABA_{C}receptor antagonist activity (e.g., compounds described in U.S. Pat. 6,632,806) may be used in the methods of the invention. It will be appreciated that pharmaceutically acceptable salts, polymorphs and variants of 3-aminopropyl-(n-butyl)-phosphinic acid (“ABPA”) and other GABA_{B} receptor antagonists above are useful in the methods of the invention.

Without intending to be bound by theory, the effects of ABPA on attention and/or hyperactivity may be related to an effect on dopaminergic transmission. ADHD may be due, at least in part, to a deficiency of dopaminergic (DA) transmission in the central nervous system, particularly the prefrontal cortex and basal ganglia, and pharmacological manipulations that facilitate dopaminergic transmission may be efficacious in treating the behavioral defects associated with ADHD. GABA_{B} antagonism may have such an effect on dopaminergic activity in the central nervous system. Administration in the substantia nigra pars reticulata (SNr) of 5-AVA, a GABA_{B} receptor antagonist, has been reported to increase dopamine release in the striatum, while administration of the GABA_{B} receptor agonist baclofen results in a decrease in striatal dopamine (Balon et al., 2002, “Indirect presynaptic modulation of striatal dopamine release by GABA_{B} receptors in the rat substantia nigra”, Neuroscience Letters 325:33-36). Systemic administration of the GABA_{B} receptor antagonist CGP 35348, was associated with a long-lasting increase in burst firing of dopamine (DA) neurons in the ventral tegmental area (VTA) suggesting that central GABA_{B} receptors may contribute to control of burst firing mode of VTA DA neurons (Erhardt et al., 2002, “GABA_{B} receptor mediated modulation of the firing pattern of ventral tegmental area dopamine neurons in vivo,” Naunyn-Schmeideberg's Arch Pharmacol. 365:173-180). Increased firing of these neurons would lead to enhanced dopamine release in brain regions that receive input from these neurons, namely the striatum and prefrontal cortex.

The specific dose, frequency and administration route for any particular patient may be varied and will depend upon a variety of factors including the activity of the specific compound employed, the metabolic stability and length of action of that compound, the age, body weight, general health, sex, diet, drug combination, and severity of the particular condition. The receptor antagonists of the invention may be administered to patients by a variety of routes, including orally, parenterally (e.g., intravenous, infusion, or implant), inhalation and topically, and may be formulated in suitable dosage unit formulations containing conventional non-toxic pharmaceutically acceptable carriers, adjuvants, excipients and vehicles appropriate for each route of administration. Exemplary excipients, diluents, and carriers are known in the art and include, for example, inert diluents; such as calcium carbonate, sodium carbonate, lactose, calcium phosphate or sodium phosphate, saline; granulating and disintegrating agents, for example, corn starch, or alginic acid; binding agents, for example starch, gelatin or acacia; lubricating agents, for example magnesium stearate, stearic acid or talc in appropriate combinations (e.g., per 200 mg ABPA:31.6 mg granular mannitol, 1.7 mg colloidal silicon dioxide, 2.4 mg magnesium stearate). Dosage schedules and routes for administration of antagonists according to the methods of the invention will vary according to the drug, the sex, age, and general health of the patient, the severity of the condition being treated and other factors.

Therapeutic formulations can be prepared by any methods well known in the art of pharmacy. See, e.g., GOODMAN AND GILMAN'S: THE PHARMACOLOGICAL BASIS OF THERAPEUTICS 10^{TH} EDITION 2001 by Louis Sanford Goodman et al., McGraw-Hill Professional; PHARMACEUTICAL DOSAGE FORMS AND DRUG DELIVERY SYSTEMS 7^{th} Edition Howard C. Ansel et al, 2004, Lippincott Williams & Wilkins Publishers; PHARMACEUTICAL CALCULATIONS 11^{th} Edition, 2001, by Mitchell J. Stoklosa et al, Lippincott Williams & Wilkins;. PHYSICAL PHARMACY: PHYSICAL CHEMICAL PRINCIPLES IN THE PHARMACEUTICAL SCIENCES 4^{th} Edition by Pilar Bustamante et al., 1993, Lea & Febiger.

The dose of a GABA_{B} receptor antagonist administered to a patient in need of treatment for an attention disorder will be a therapeutically effective amount. Generally the dosage for a GABA_{B} receptor antagonist will be between about 0.001 to 1000 mg per kg patient body weight per day, and more often between about 0.01 and 25 mg/kg/d, which can be administered in single or multiple doses. As used herein, reference to a therapeutically “effective amount” or a “sufficient amount” of an antagonist means an amount sufficient to effect a desired biological effect, such as beneficial results and, as such, an “effective amount” depends upon the context in which it is being applied. In general, an effective amount is an amount sufficient to result in an improvement in attention and/or reduction of hyperactivity when administered to a subject in need of such improvement. Alternatively, an effective amount is an amount sufficient to antagonize a GABA_{B} receptor in the subject. An effective amount can be administered in one or more administrations. As used in this context, an improvement is a statistically significant change in attention and/or hyperactivity in a subject, as assessed by a clinician or the subject, so that the level of attention and/or hyperactivity is more like that of a normal subject not diagnosed with an attention disorder.

In accordance with the invention, 3-aminopropyl-(n-butyl)-phosphinic acid can be administered via a variety of routes and can be administered at any effective dose. ABPA is orally active and is generally administered orally for convenience. As noted above, the dose administered will vary and depend on a variety of factors. In one embodiment, the dose is between about 1 mg and about 3000 mg per day, and more often between about 1 mg and 2000 mg per day. For example and not limitation, suitable dosing configurations include single oral doses ranging from 10 to 2100 mg, multiple oral doses ranging from 300 to 1200 mg t.i.d. (i.e., 900 to 3600 mg per day) and single intravenous doses ranging from 315 to 1800 mg. In one embodiment ABPA is administered orally at a dosage less that 1800 mg/day, such as less that 1500 mg/day or less than 1000 mg/day. A treatment regimen can comprise as little as a single administration of antagonist, but more often will be for a period of several weeks, and often for months or years. In some embodiments, ABPA is administered for at least about two weeks, at least about four weeks, or at least about eight weeks. The antagonist may be administered daily (in a single or multiple doses) or less frequently. For example, for illustration and not limitation, the antagonist may be administered at least once per week for one, two, three or more than three weeks; daily for at least one week; daily over a period of at least eight weeks, or according to other schedules.

In one embodiment, a daily dose of from 50 mg to 4000 mg of 3-aminopropyl-(n-butyl)-phosphinic acid is administered for at least about two, and alternatively at least about eight weeks. As is understood in the art, treatment can be suspended temporarily if toxicity is observed, or for the convenience of the patient, and then resumed, without departing from the scope of the invention.

IV. Patient Populations and Treatment Methods

In one aspect, the invention provides methods for treatment of an attention disorder by administering an effective amount of a GABA_{B} receptor antagonist to a subject in need of such treatment. “Treatment” has its ordinary meaning and refers to an approach for obtaining beneficial or desired results, including clinical results. For purposes of this invention, beneficial or desired clinical results include, but are not limited to, alleviation or amelioration of one or more symptoms, diminishment of extent of disease, stabilized (i.e., not worsening) state of disease, preventing spread of disease, delay or slowing of disease progression, amelioration or palliation of the disease state, and remission (whether partial or total).

Subjects

The subject in need of treatment is generally a human diagnosed as having an attention disorder. In various embodiments of the invention the attention disorder may be ADD or ADHD (which may be Combined Type, Predominantly Inattentive Type; or Predominantly Hyperactive-Impulsive Type ADHD).

In one embodiment, the subject is a child (i.e., not older than 12 years of age). In one embodiment, the subject is an adolescent (i.e., from 13 to 17 years of age). In one embodiment, the subject is an adult (i.e., at least 18 years of age). In some embodiments, the subject is not older than 40 years of age, not older than 15 years of age or not older than 12 years of age. In one embodiment the subject is from 10 to 40 years of age. In one embodiment the subject is a man or a woman from 19 to 55 years of age.

In one embodiment, the subject is not diagnosed with and/or does not suffer from mild cognitive impairment (MCI). In one embodiment the subject is not diagnosed with and/or does not suffer from Alzheimer's disease (AD). In one embodiment, the subject is not diagnosed with and/or does not suffer from a condition independently selected from: a neurodegenerative disease, stress-induced neurodegeneration, a motor neuron disease, amyotrophic lateral sclerosis, spinal muscular atrophy, post-polio syndrome, Parkinson's disease or syndromes, suppression of immune responses following CNS tissue graft; Huntington's chorea, a basal ganglia disorder; neuroinflammation, multiple sclerosis, inflammatory hyperalgesia, severe depression, schizophrenia, peripheral neuropathy; a convulsive state (e.g. resulting from epilepsy and excitotoxic/ischemic damages) and/or an anxiety disorder. In one embodiment the subject is not diagnosed with and/or does not suffer from a spinal cord injury and/or head trauma and/or traumatic brain injury.

In clinical trials, patients with MCI receiving ABPA also showed significant improvements in psychomotor speed as assessed by CANTAB tests, indicating that compounds with GABA_{B} receptor antagonist activity may be administered to subjects to improve psychomotor speed.

Monitoring

In one aspect, the invention provides a method for treating an attention disorder by (a) administering a GABAB receptor antagonist, e.g., ABPA to a subject diagnosed with a disorder of attention and (b) determining whether one or more manifestations of the disorder are reduced in the subject following administration (i.e., following a course of therapy with the agonist).

As noted above, it is within the skill of medical professionals to diagnose attention disorders. Manifestations of attention disorders include, but are not limited to, behaviors used in the diagnosis of ADHD and ADD such as: a. often careless and inattentive to details and makes careless mistakes in schoolwork, work or other activities; b. Often has difficulty sustaining attention in tasks or play activities; c. Often does not seem to listen when spoken to directly; d. Often does not follow through on instructions and fails to finish schoolwork, chores, or duties in the workplace (not due to oppositional behavior or failure to understand instructions); e. Often has difficulty organizing tasks and activities; f. Often avoids, dislikes or is reluctant to engage in tasks that require sustained mental effort, such as schoolwork or homework; g. Often loses things necessary for tasks or activities e.g., toys, school assignments, pencils, books, or tools; h. Is often easily distracted by extraneous stimuli; i. Is often forgetful in daily activities; j. often fidgets with hands or feet or squirms in seat; k. often leaves seat in classroom or in other situation in which remaining seated is expected; I. often runs about or climbs excessively in situations in which it is inappropriate (in adolescence or adults, may be limited to subjective feelings of restlessness); m. often has difficulty playing or engaging in leisure activities quietly; n. is often “on the go” or often acts as if “driven by a motor;” o. often talks excessively; p. often blurts out answers before questions have been completed; q. often has difficulty awaiting turn; r. often interrupts or intrudes on others, e.g., butts into conversations or games. Manifestations (a)-(i) are sometimes considered symptoms of inattention; manifestations (j)-(o) are sometimes considered symptoms of hyperactivity; and manifestations (p)-(r) are sometimes considered symptoms of impulsivity. Usually a diagnosis is made based on persistence of symptoms for at least 6 months. Although many individuals present with symptoms of both inattention and hyperactivity-impulsivity, there are individuals in whom one or the other pattern is predominant. An ADHD subtype can be specified based on the predominant symptom pattern for the past 6 months. Combined Type ADHD is characterized by six or more symptoms of inattention and six or more symptoms of hyperactivity-impulsivity that have persisted for at least 6 months; Predominantly Inattentive Type ADHD is characterized by six or more symptoms of inattention but fewer than six symptoms of hyperactivity-impulsivity that have persisted for at least 6 months; and Predominantly Hyperactive-Impulsive Type or ADHD is characterized by six or more symptoms of hyperactivity-impulsivity but fewer than six symptoms of inattention) that have persisted for at least 6 months.

In one aspect, the invention provides a method for treating an attention disorder by (a) administering a GABA_{B} receptor antagonist, e.g., ABPA to a subject diagnosed with a disorder of attention and (b) determining whether improvement in a quantitative measure of attention occurs following administration (i.e., following a course of therapy with the agonist). An example of a quantitative measure of attention is performance on a test for attention. A number of tests for attention are known and include the ADHD Rating Scale (ADHDRS or ADHD RS-IV), the IOWA Conners Scale, the Conners Rating Scales-Revised (CRS-R), the Conners' Adult ADHD Rating Scales (CAARS), and select tests of the CANTAB. See, e.g., Collett et al., 2003, “Ten-Year Review of Rating Scales. V: Scales Assessing Attention-Deficit/Hyperactivity Disorder”, J. American Academy of Child & Adolescent Psychiatry 42:1015-37; DuPaul et al., 1998, ADHD Rating Scale-IV: Checklist, Norms, and Clinical Interpretation. New York: Guilford; Pelham et al, 1989, “Normative data on the IOWA Conners Teacher Rating Scale”, J. Clinical Child Psychology 18:259-62; Conners, 1997, Conners' Rating Scales-Revised Technical Manual. North Tonawanda, NY: Multi-Health Systems; Simpson and Plosker, 2004, “Atomoxetine: a review of its use in adults with attention deficit hyperactivity disorder”, Drugs 64:205-222; Robbins et al., 1994, “Cambridge Neuropsychological Test Automated Battery (CANTAB): A factor analytic study of a large sample of normal elderly volunteers,” Dementia 5:266-281.

In some embodiments, an improvement in a quantitative measure of attention and/or a reduction in a manifestation of an attention disorder (e.g., hyperactivity) can be observed in the subject after two-weeks treatment, after four weeks treatment, after eight weeks treatment and/or after 25 weeks treatment.

V. Combination Therapy

The methods of the present invention encompass the co-administration of a GABA_{B} receptor antagonist (e.g., ABPA) in combination with one or more other agents (drugs) useful for treatment of an attention disorder. Drugs are administered to a subject “in combination” when the drugs are administered as part of the same course of therapy. In this context, “a course of therapy” refers to administration of combinations of drugs believed by the medical professional to work together additively, complementarily, synergistically, or otherwise to produce a more favorable outcome than that anticipated for administration of a single drug. A course of therapy can be for one or a few days, but more often extends for several weeks. Combinations of drugs may be administered simultaneously (co-administered) or according to different administration schedules, and in the same or separate formulations. Agents commonly used for treatment of attention disorders and which may be administered in combination with a receptor antagonist include dextroamphetamine sulfate, sustained release (Dexedrine™); methylphenidate hydrochloride (Ritalin™); pemoline (Cylert™); atomoxetine hydrochloride (Strattera™). Other agents useful for treatment of attention disorders include nicotine receptor agonists, α1 or α2 adrenergic receptor ligands, D2 receptor agonists, 5HT1A receptor agonists, and cholinesterase inhibitors such as compounds listed in U.S. patent publication 2002/0016334, incorporated herein by reference.

VI. Advertising

another aspect, the invention provides a method entailing (a) advertising the use of ABPA, or another GABA_{B} receptor antagonist, for treatment of ADD or ADHD, and (b) selling ABPA, or another GABA_{B} receptor antagonist, to individuals for use for treatment of ADD or ADHD. It will be appreciated that selling to “individuals” includes sales to corporate persons (corporations) and the like (for example, sales to a medical facility for distribution to patients for treatment of ADD or ADHD.

VII. Screening

In a related aspect, the invention provides methods for identification of agents useful for treatment of a disorder of attention by (1) identifying an agent as a GABA_{B} receptor antagonist and (2) determining whether the antagonist has an attention enhancing effect in a mammal. Preferably the mammal is a non-human animal useful as a model for an attention disorder. Examples of such models include the Naples-High Excitability (NHE) rat (see, e.g., Viggiano et al., 2003, “Behavioural, pharmacological, morpho-functional molecular studies reveal a hyperfunctioning mesocortical dopamine system in an animal model of attention deficit and hyperactivity disorder” Neurosci Biobehav Rev. 27:683-9); Spontaneously Hypertensive Rats (SHR; see Leo et al., 2003, “Altered midbrain dopaminergic neurotransmission during development in an animal model of ADHD” Neurosci Biobehav Rev. 27:661-9); and models described in Davids et al., 2003, “Animal models of attention-deficit hyperactivity disorder” Brain Res Rev 42:1-21, and references cited therein. Other useful models include genetic models known in the art (e.g., the dopamine transporter knock-out mouse; the Coloboma mutant mouse; and the Acallosal mouse strain I/LnJ); Neurotoxin-exposed animals (e.g., juvenile rats with neonatal 6-hydroxydopamine brain lesions; rats exposed to neonatal hypoxia, developmental cerebellar stunting or X-ray damage of hippocampus; and animals exposed to environmental toxins such as polychlorinated biphenyls) and others (e.g., hyposexual male rats and animals selected from a general population based on deficit in five-choice serial reaction time task). In one embodiment the method includes the further step of comparing the attention enhancing effect of the agent with that of ABPA. Generally, an agent that is at least as effective, on a molar basis, as ABPA is preferred.

In a related embodiment, the invention provides methods for identification of agents useful for treatment of a disorder of attention by (1) identifying an agent as a GABA_{B} receptor antagonist and (2) determining whether the antagonist has an attention enhancing effect in a mammal. Compounds that are GABA_{B} receptor antagonists can be identified by reference to the scientific literature or using assays known in the art (see, e.g., U.S. Pat. No. 6,632,806).

In a related embodiment, the invention provides methods for identification of agents useful for treatment of a disorder of attention by (1) identifying an agent as a GABA_{B} receptor antagonist and (2) determining whether the antagonist reduces hyperactivity in a mammal.

### VII. EXAMPLES

Human Clinical Trial

A clinical trial was initiated to determine the effect of the GABA_{B} receptor antagonist, ABPA, on specific tests of cognition in non-demented older individuals with diminished memory function who met prospective criteria for mild cognitive impairment (MCI). Patients aged 60-85 with MCI enrolled in this double-blind, randomized trial. Patients were randomized to ABPA (600 mg tid) or placebo for 8 weeks. Verbal, paper and computer tests of memory and cognition were made by trained assessors at baseline (before administration of ABPA or placebo) and after 2, 5, and 8 weeks of treatment. Among these were selected tests from the Cambridge Neuropsychological Test Automated Battery (CANTAB), a computerized battery of tests for neuropsychological evaluation (Robbins et al., 1994, “Cambridge Neuropsychological Test Automated Battery (CANTAB): A factor analytic study of a large sample of normal elderly volunteers,” Dementia 5:266-281). Tests assessing memory, attention and psychomotor speed were administered to the patient by a neuropsychologist or equivalent via a computer with a touch screen, thus testing the individual's cognitive performance rather than ability to use a computer. CANTAB tests have been proven to be highly sensitive and specific in differentiating patients with a number of neurological conditions from each other and from normal controls.

The results were as follows. 110 patients were randomized; 75 were administered ABPA and 35 were administered placebo (56% men; mean age: 74 years). Significant differences and trends were observed in a number of endpoints including two distinct tests of attention, the Five Choice Reaction Time test and the Rapid Visual Information Processing test.

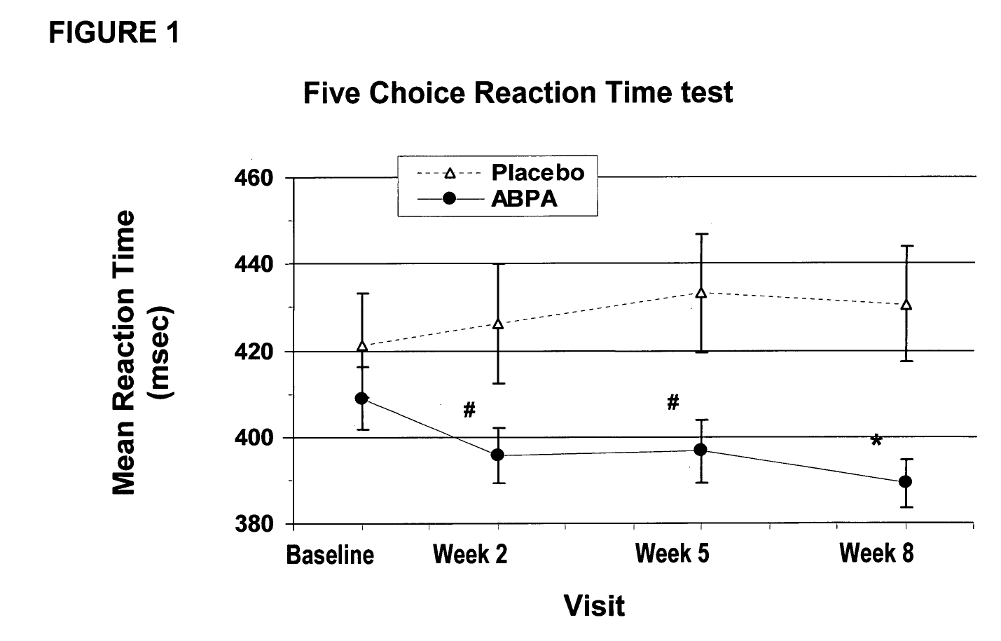

The Five Choice Reaction Time test measures the speed of response to the appearance of a visual stimulus. Patients rested their hand on a press-pad placed on the desk directly in front of the computer screen, holding down the press-pad until a brief yellow flash appeared in 1 of the 5 circles located at various positions around the screen. As quickly as possible, the patients were required to touch the circle where the yellow flash had appeared. To perform this task, the patient must attend to all five of the positions where the yellow flash could appear. The computer recorded 2 measures of response speed: reaction time, which was the time taken to lift the hand from the press-pad, and movement time, which was the latency from press-pad release to the time that the screen was touched. Mean reaction time on this task was significantly decreased after 8 weeks of treatment with ABPA compared with treatment with placebo (FIG. 1). Compared to baseline measures, the mean reaction time at 8 weeks was decreased in the ABPA group by −19.7 milliseconds. Meanwhile, the placebo group showed an increase in the reaction time by +9.4 milliseconds at 8 weeks, a statistically significant difference between the two treatment groups (p=0.002). Decreased reaction time with ABPA and increased reaction time with placebo were also seen at Week 2 and Week 5, and the differences between the treatment groups approached statistical significance at each time point (p=0.074 and 0.070, respectively).

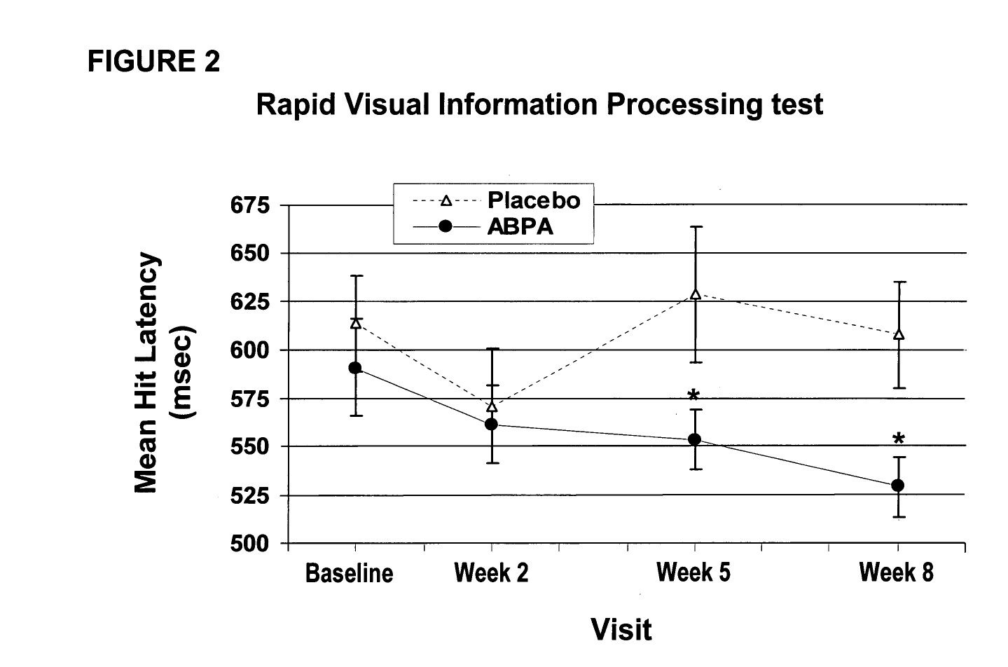

The Rapid Visual Information Processing (RVIP) test is a test of sustained attention with a small working memory component. Digits were presented singly on a computer screen at a rate of 100 digits per minute. The patient was asked to detect specific sequences of digits (e.g., 2, 4, 6 or 5, 7, 9) in this pseudo-random string of numbers. Responses were recorded with a button press. Reaction time, or mean hit latency, for all correct responses was recorded, as were additional signal detection measures (e.g., total hits, total misses and total false alarms). Administration of the anti-ADHD agent methylphenidate has been shown to improve performance in the RVIP test in patients diagnosed with adult ADHD (Turner et al., 2005, “Neurocognitive affects of methylphenidate in adult attention-deficit/hyperactivity disorder” Psycho-pharmacology 78:286-95) In the clinical trial, the group administered ABPA showed significantly greater improvement than the placebo group at Week 5 and Week 8 relative to Baseline (p=0.03 and p=0.02, respectively; FIG. 2). The mean change from Baseline was −37.3 msec in the ABPA group and +15.0 msec in the placebo group at Week 5, and −61.6 msec in the ABPA group and −5.9 msec in the placebo group at Week 8.

These results illustrate that ABPA had a beneficial effect on attentional processes and psychomotor speed in these elderly patients.

Animal Models

Attention-deficit/hyperactivity disorder (ADHD) is a major neuropsychiatric disorder characterized by abnormalities in attentional processes, hyperactivity and impulsivity (Davids et al., 2003, “Animal models of attention-deficit hyperactivity disorder,” Brain Research Reviews 42:1-21). The coloboma mutant mouse has been identified as an animal model for examining the neurobiological basis of at least one behavioral aspect of ADHD: hyperactivity (Wilson, 2000, “Coloboma mouse mutant as an animal model of hyperkinesis and attention deficit hyperactivity disorder,” Neuroscience and Biobehavioral Reviews 24:51-57). Tests were conducted to determine whether administration of the GABA_{B} receptor antagonist ABPA could normalize the hyperactivity of coloboma mice.

The coloboma mouse was produced by neutron irradiation and the resulting mutation is a chromosomal deletion mapped to mouse chromosome 2. Although the coloboma mutation (Cm) is early embryonic lethal when homozygous, mice heterozygous for Cm (genotype, Cm/+) exhibit a number of behavioral, neurophysiological and developmental deficits that model various impairments characteristic of human ADHD. These mice show robust spontaneous hyperactive locomotor behavior, learning deficits, and delays in neurodevelopment (Raber et al., 1997, “Coloboma hyperactive mutant mice exhibit regional and transmitter-specific deficits in neurotransmission” Journal of Neurochemistry 68:176-86; Hess et al., 1996, “Mouse model of hyperkinesis implicates SNAP-25 in behavioral regulation,” Journal of Neuroscience 16:3104-11; Heyser et al., 1995, “Coloboma hyperactive mutant exhibits delayed neurobehavioral developmental milestones,” Brain Research: Brain Developmental Research 89:264-69). The behavioral abnormalities seen in these mutant mice are believed to arise, at least in part, from the deletion of the gene Snap which encodes SNAP-25, a key component of the neurotransmitter release machinery (Hess et al., 1996). Alterations in this gene have been identified as playing a role in human ADHD (Barr et al., 2000, “Identification of DNA variants in the SNAP-25 gene and linkage study of these polymorphisms and attention-deficit hyperactivity disorder,” Molecular Psychiatry 5:405-409). Mutation of SNAP-25 leads to profound disruption of dopamine release, especially in brain regions such as the dorsal striatum. This deficiency in dopaminergic neurotransmission may underlie the behavioral hyperactivity and impaired information processing observed in this mutant mouse.

The hyperactivity displayed by these mice is, on average, 3- to 10-fold higher than their wildtype littermates, with considerable individual variation. The high degree of variability suggests a loss of control of locomotor activity in these mice rather than a simple increase in basal motor behavior. Interestingly, the spontaneous hyperactivity exhibited by Cm/+ mice can be attenuated by application of compounds that are effective anti-ADHD drugs in humans. For example, activity levels of coloboma mice as measured by an open field test were reduced by low doses of dextro-amphetamine (d-amphetamine; Hess et al., 1996; Wilson et al., 2000; WO 03/007956 Al; US 2005/0004105 Al). The same dose of d-amphetamine administered to wildtype littermates produced profound increases in activity. However, another stimulant ADHD medication methylphenidate failed to normalize the hyperactivity in Cm mutant mice, instead increasing locomotor activity in both mutant and wildtype mice in a dose-dependent manner. Atomoxetine, a non-stimulant drug effective in treating human ADHD reduces hyperactivity in coloboma mice without affecting activity levels in wildtype mice.

Animals

Male and female mice heterozygous for the coloboma spontaneous mutation (C3H/HeSnJ-Cm/J) and wild type (C3H/HeSNJ) mice from Jackson Laboratories (Bar Harbor, Me.) were used in this study. Upon receipt, mice were assigned unique identification numbers (tail marked) and were housed in polycarbonate cages with filter tops. All mice were acclimated to the colony room for at least two weeks prior to testing. During the period of acclimation, mice were examined on a regular basis, handled, and weighed to assure adequate health and suitability. Mice were maintained on a 12/12 light/dark cycle. Chow and water were provided ad libitum for the duration of the study. In each test, animals were randomly assigned across treatment groups and balanced by age and by gender. Animals were not disturbed between test days.

Open Field Testing

The open field test (OF) is used to assess both anxiety-like behavior and motor activity. The open field chambers are Plexiglas square chambers (27.3×27.3×20.3 cm; Med Associates Inc., St Albans, Vt.) surrounded by infrared photobeam sources (16×16×16). The enclosure was configured to split the open field into a center and periphery zone and the photocell beams were set to measure activity in the center and in the periphery of the OF chambers. Animals having higher levels of anxiety or lower levels of activity tend to stay in the corners of the OF enclosures. On the other hand, mice that have high levels of activity and low levels of anxiety tend to spend more time in the center of the enclosure. Distance traveled is measured from consecutive beam breaks. Measures of peripheral or central distance covered during locomotion were used as an index of activity.

Drug Treatment

ABPA (0.3, 3 and 30 mg/kg) and atomoxetine (0.1 mg/kg) were dissolved in saline and administered i.p. at the injection volume of 10 ml/kg. Mice were pretreated with saline, ABPA, or atomoxetine for 30 min, after which their behavior was recorded in the OF chambers for a 60-min test session. At the end of each open field test session the OF chambers were thoroughly cleaned with Clorox wipes. Mice were returned to their home cage after testing and then to the colony room. Mice were retested every week. The washout period between tests ranged from 3-7 days. Data were analyzed by analysis of variance (ANOVA), followed by Fisher PLSD post-hoc analysis when appropriate. An effect was considered significant if p<0.05. Outliers that fell above and below two standard deviations away from the mean were removed from the final analysis.

ABPA Reduced Spontaneous Hyperactivity in the Coloboma Mouse:

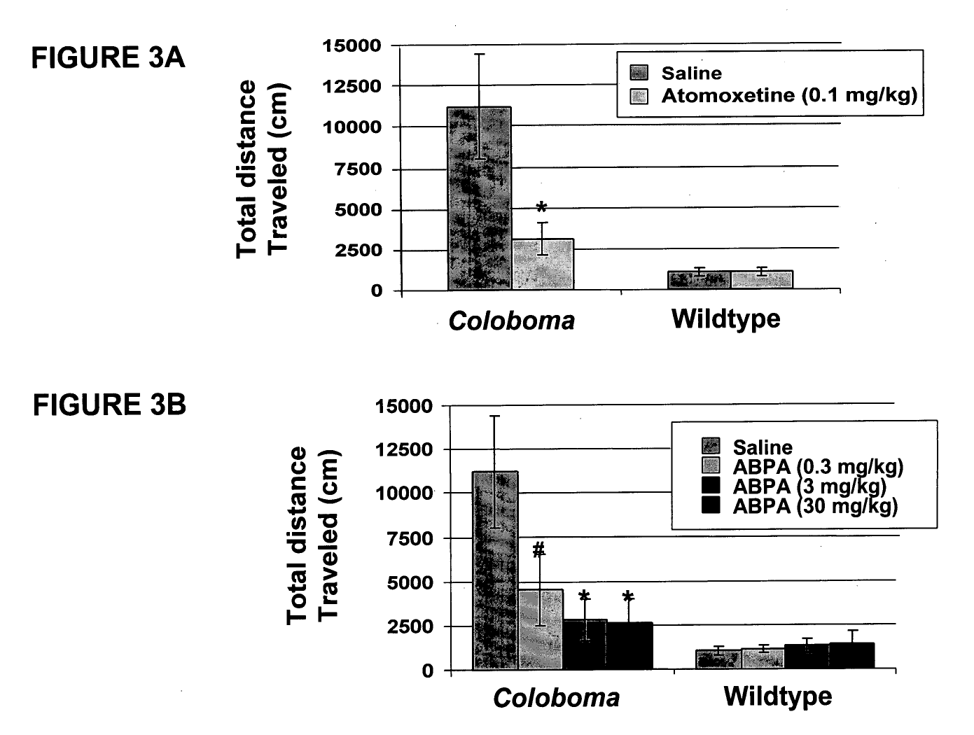

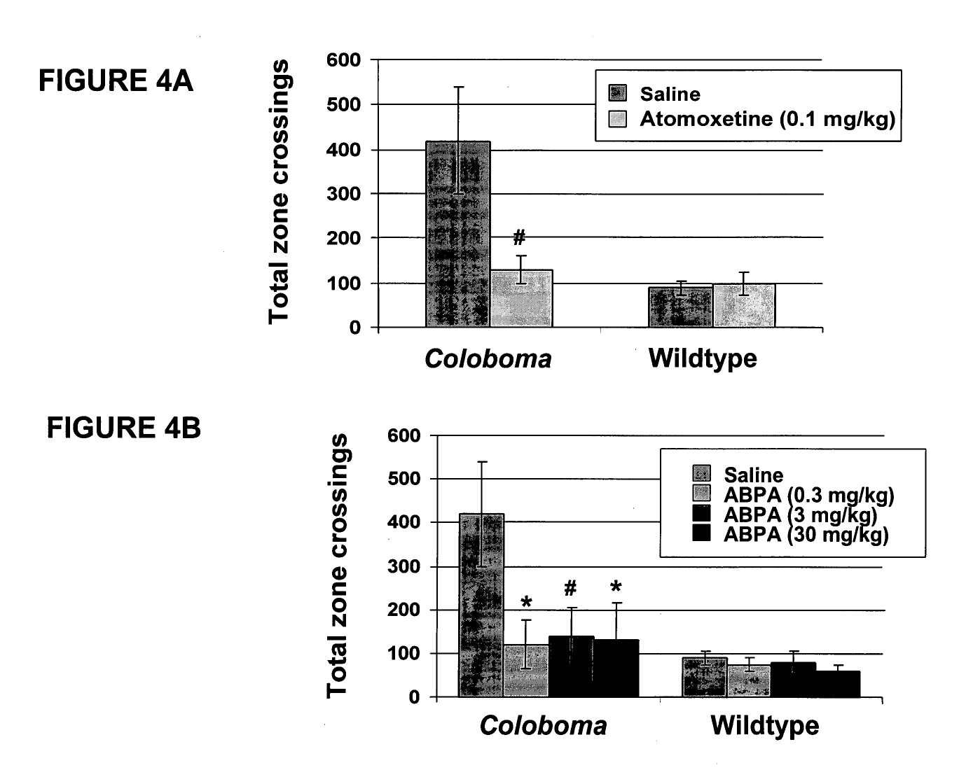

The effects of atomoxetine and ABPA on locomotor activity levels in coloboma and wildtype mice are presented in FIGS. 3 and 4. Since the effects of all drugs reached a stable response by 20-30 min following the start of the experiment, the data was analyzed in 2 time periods: 1) total 60 min test session and 2) the last 30 min of the test session. The quantified data over the final 30 min time period are presented. Repeated measure ANOVA found a significant genotype and drug interaction over both time periods. As measured by total distance traveled, the coloboma mice had roughly 6- to 10-fold higher saline-induced activity compared to the wildtype mice (11,246±3213 centimeters vs. 1035±252 centimeters, FIGS. 3A and 3B). Over the 30-min period, administration of 0.1 mg/kg atomoxetine to Cm mutant mice significantly decreased total distance traveled relative to saline-treated Cm mutants (FIG. 3A; p=0.03). Administration of the GABA_{B} receptor antagonist ABPA had a similar effect on activity in the coloboma mutant mice. Treatment with ABPA (3 and 30 mg/kg) significantly decreased locomotor activity levels of the coloboma mice only, with no effect on the activity of wildtype controls (FIG. 3B; p=0.03 for both doses). The lowest dose of ABPA (0.3 mg/kg) showed a strong trend to decreasing the activity of the coloboma mice (p=0.07).

Saline-treated mutant mice also crossed zones more frequently (center-to-periphery and vice versa) than did their saline-treated wildtype littermates (420±120 zone crosses vs. 90±15 zone crosses, FIGS. 4A and 4B). Over the final 30-min period, 0.1 mg/kg atomoxetine (FIG. 4A) and 3 mg/kg ABPA (FIG. 4B) showed a strong trend to decreasing the zone crossings in Cm mutants (p=0.06 for both), while ABPA at 0.3 or 30 mg/kg (FIG. 4B) produced a significant decrease in the frequency of zone crossings in Cm mutants to within the range of saline-treated wildtype mice (p=0.04 for both doses).

In parallel experiments, low doses of d-amphetamine (0.5, 1 and 4 mg/kg) unexpectedly did not reduce activity in the coloboma mice, but 4 mg/kg d-amphetamine did reduce the frequency of rearing by the mice. D-amphetamine did increase activity in control (wildtype) mice, but had no effect on rearing frequency.

While the present invention has been described with reference to the specific embodiments thereof, it should be understood by those skilled in the art that various changes can be made and equivalents can be substituted without departing from the scope of the invention. In addition, many modifications can be made to adapt a particular situation, material, composition of matter, process, process step or steps, to achieve the benefits provided by the present invention without departing from the scope of the present invention. All such modifications are intended to be within the scope of the claims appended hereto.

All publications and patent documents cited herein are incorporated herein by reference as if each such publication or document was specifically and individually indicated to be incorporated herein by reference. Citation of publications and patent documents is not intended as an indication that any such document is pertinent prior art, nor does it constitute any admission as to the contents or date of the same.

1. A method for treatment of an attention disorder comprising administering an effective amount of a GABA_{B} receptor antagonist to a subject in need of such treatment.

2. The method of claim 1 wherein the disorder is Attention Deficit Disorder (ADD) or Attention-Deficit/Hyperactivity disorder (ADHD).

3. The method of claim 2 wherein the antagonist is 3-aminopropyl-(n-butyl)-phosphinic acid (ABPA).

4. The method of claim 3 wherein the subject is a child.

5. The method of claim 3 wherein the subject is an adolescent.

6. The method of claim 3 wherein the subject is an adult.

7. The method of claim 6 wherein the subject is not diagnosed with Mild Cognitive Impairment (MCI) or Alzheimer's disease (AD).

8. The method of claim 1 wherein the GABA_{B} receptor antagonist is administered in combination with a second treatment for attention deficit.

9. The method of claim 8 wherein the other treatment is selected from the group consisting of dextroamphetamine, methylphenidate, pemoline and atomoxetine hydrochloride.

10. The method of claim 1 wherein the antagonist is administered at least once per week for one, two, three or more than three weeks.

11. The method of claim 10 wherein the antagonist is daily for at least one week.

12. The method of claim 10 wherein the antagonist is administered over a period of at least eight weeks.

13. The method of claim 1 comprising (a) diagnosing a disorder of attention in a subject, (b) administering a GABA_{B} receptor antagonist to the subject and (c) determining whether one or more manifestations of the disorder are reduced in the subject.

14. The method of claim 1 comprising (a) administering a GABA_{B} receptor antagonist to the subject diagnosed with a disorder of attention and (b) determining whether one or more manifestations of the disorder are reduced in the subject.

15. The method of claim 14 wherein the disorder is Attention Deficit Disorder (ADD) or Attention-deficit/hyperactivity disorder (ADHD).

16. The method of claim 15 wherein the antagonist is 3-aminopropyl-(n-butyl)-phosphinic acid (ABPA).

17. The method of claim 1 comprising (a) diagnosing an ADD or ADHD in a subject, (b) administering a GABA_{B} receptor antagonist to the subject for a period of time, and (c) detecting an improvement in a quantitative measure of attention following step (b).

18. The method of claim 1 comprising (a) administering a GABA_{B} receptor antagonist for a period of time to a subject diagnosed with a disorder of attention and (b) detecting an improvement in a quantitative measure of attention following step (a).

19. A method for treatment of an attention disorder comprising administering a daily dose of from 10 mg to 2100 mg of 3-aminopropyl-(n-butyl)-phosphinic acid (ABPA) for at least eight weeks to a subject in need of treatment for Attention Deficit Disorder (ADD) or Attention-deficit/hyperactivity disorder (ADHD).

20. A method for enhancing attention and/or reducing hyperactivity in a subject comprising administering a GABA_{B} receptor antagonist to the subject.

21. The method of claim 20 wherein the antagonist is 3-aminopropyl-(n-butyl)-phosphinic acid (ABPA).

22. A method for identification of an agent useful for treatment of a disorder of attention comprising (1) identifying an agent as a GABA_{B} receptor antagonist and (2) determining whether the antagonist has an attention enhancing effect in a mammal.
